(19) United States
(12) Patent Application Publication
(10) Pub. No.: US 2011/0263008 A1
(43) Pub. Date: Oct. 27, 2011

(54) Cell Culture Real-Time Observation System

(75) Inventors: KER-WIN WANG, Changhua County (TW); NANCY M. WANG, Changhua County (TW); CHUIAN-FU KEN, Changhua County (TW)

(73) Assignee: NATIONAL CHANGHUA UNIVERSITY OF EDUCATION, Changhua County (TW)

(21) Appl. No.: 12/914,417

(22) Filed: Oct. 28, 2010

(30) Foreign Application Priority Data
Apr. 26, 2010 (TW) 099113150

(51) Int. Cl. C12M 3/00

(52) U.S. Cl. 435/288.7; 435/292.1; 435/303.1

(57) ABSTRACT

The present invention discloses a cell culture realtime observation system comprising a plate, a culture well, an image capture device, at least one container, an evaporation device, a first micro channel, a second micro channel and a transmission unit. The culture well is disposed on the plate, and the image capture device is disposed between the plate and the culture well. The container is disposed on the plate and at a side of the culture well, and the evaporation device is disposed at the other side of the culture well. The container and the culture well are connected by the first micro channel, and the culture well and the evaporation device are connected by the second micro channel. The transmission unit is electrically connected to the image capture device.

### FIELD OF THE INVENTION

The present invention relates to an observation system, and more particularly, to a cell culture realtime observation system.

### BACKGROUND OF THE INVENTION

Infertility is the inability of a couple to become pregnant for more than two years after unprotecting sexual intercourse. According to statistics, one in seven couples may have difficulty conceiving. Infertility can be caused by many different factors, such as male and female factors. The male factors and the female factors are accounted for 3040% and 6070% in all infertility factors, respectively. The male factors may comprise abnormal semen characteristics, such as reduction in the number of sperms, sperm dysfunction, decreased sperm mobility, and etc. The major factors leading to female infertility include tubal obstruction, autoimmune diseases, endometriosis or ovulatory dysfunction.

Most of infertility cases are treated with medication or surgery. Improvements in fertility treatments have made it possible for many women to become pregnant. These advanced technologies include intrauterine insemination (IUI), in vitro fertilization (IVF), and other similar procedures. For the IUI, females will take a medicine or inject drug to stimulate ovulation firstly. Sperm, collected from a man, is washed and concentrated to remove inactive sperms and impurities. The sperm with the best quality is placed into the womb by a tube to reach the fertilization. During the IVF procedure, the fertilization of an egg occurs outside the body. Eggs are removed from the female ovaries by a transvaginal ultrasound and fertilized by sperm microinjection techniques to culture embryos. The fertilized embryo or embryos is/are then selected and put back inside the female's body.

One of infertility treatment outcomes depends on the cultivation and screening of fertilized embryos. It is necessary to have a good culture environment during culture period, and to effectively decrease negative affects resulted from artificial interference or environmental factors. Generally, conventional embryo culture is that an embryo is cultured in a culture dish. Because the culture dish is not a dynamic environment, the medium in the culture dish will be always replaced with new medium. Thus, culture variables, such as the temperature, osmotic pressure and pH values of the medium or air quality, may be increased. Furthermore, when the embryo is detected by a microscope, the temperature of parts of the embryo may be increased due to the focus-light of the microscope, so as to affect the development and growth of the embryo. When the embryo containing in the culture dish is taken out from an incubator to take a picture, the embryo development may be influenced because of temperature shock or physical stress. Conventional cell culture devices also have many disadvantages as following examples: (1) the conventional cell culture devices are expensive, space-consuming, and do not dispose into an incubator. (2) The manufacturing procedure of the conventional cell culture devices and the micro-fluid system thereof are complicated. (3) The conventional cell culture devices do not be sterilized, such that bacteria, virus or other toxicants adhere onto the commercial cell culture devices. (4) The conventional cell culture devices do not provide realtime observation. (5) The controlled range of the flow velocity of the medium in the conventional cell culture devices is very small. (6) The fluid shear stress, temperature, pH values and osmotic pressure thereof will be changed by conventional micro-fluid pumps while removing metabolites.

### SUMMARY OF THE INVENTION

In view of the aforementioned drawbacks in existing skills or techniques, an object of the present invention is to provide a cell culture realtime observation system, so as to not only observe the growth, activity and replication of a cell in real time for a user, but also detect variables affecting on cell culture.

To achieve the above objects, the cell culture realtime observation system comprises a substrate, a culture well, an image capture device, at least one container, an evaporation device, a first micro-channel, a second micro-channel and a transmitting unit. The culture well is disposed on the substrate and has an opening capable of closing for placing a cell in the culture well. The image capture device is disposed between the substrate and the culture well to capture the image of the cell. The at least one container is disposed on the substrate and at a side of the culture well for containing a culture medium. The evaporation device is disposed on the substrate and at the downstream of the culture well, and a surface of the evaporation device has a plurality of evaporation holes. The at least one container is connected to the culture well by the first micro-channel. The culture well is connected to the evaporation device by the second micro-channel. The transmission unit is electrically connected to the image capture device and transmits the image captured by the image capture device to an electronic device. The culture medium is flowed through the at least one container, the culture well and the evaporation device via the first micro-channel and the second micro-channel to immerse the cell in the culture medium. When the culture medium is flowed into the evaporation device, the culture medium generates an evaporative driving force and is flowed slowly in the first micro-channel and the second micro-channel. When the image of the cell is captured by the image capture device, the captured image can be transmitted to an electronic device through the transmitting unit. Thus, a user can observe the cell in real time during cultivation.

In addition, the cell culture realtime observation system of the present invention further comprises a package mechanism. The culture well, the image capture device, the at least one container, the evaporation device, the first micro-channel and the second micro-channel are packaged within the package mechanism, and the transmission unit is disposed at an end of the package mechanism for transmitting the image to the electronic device. A steady, strobe or programmable light source device may be disposed on the package mechanism corresponding to the culture well, and the light source device radiating light to the cell. The light can provide the captured image being cleaner.

The cell culture realtime observation system according to the present invention provide one or more of the following advantages:

(1) The evaporation device of the present invention has a plurality of evaporation holes, such that a user can control the sizes of the plurality of evaporation holes to control the evaporative driving force of the culture medium. The velocity and hydraulic pressure thereof can be stable, and the change of fluid shear stress or impact stress can be avoided. Therefore, it is unnecessary to dispose expensive mechanical and microelectromechanical (MEM) pumps.

(2) The light source device of the present invention does not affect the development and growth of cells or organisms, and the development rate of the cells can be promoted by different wavelength light irradiating from the light source.

(3) The cell culture realtime observation system of the present invention can be placed in an incubator, such that the cells can grow stably. The cell growth can be detected synchronously through the image capture device and the transmitting unit to overcome the uncertainty of the external environment.

(4) The cell culture realtime observation system of the present invention can be sterilized by immersing in ethanol, an ethylene oxide (E.O.) sterilization, a radiation sterilization or an ozone sterilization, and therefore all cell contaminations resulted from environment and user factors can be prevented.

(5) The cell culture realtime observation system of the present invention has low cost and small size characteristics. Additionally, the cell culture realtime observation system can be designed in disposability or reusability to further decrease usage costs.

(6) The image capture device with low costs, such as CMOS image capture device, is used as an image sensor in the cell culture realtime observation system of the present invention, and the convenience and accuracy of detecting cell growth are increased on the medicine science. The cell culture realtime observation system of the present invention can be applied to infertility treatments, cell physiology, single cell culture and research, the research of cell cluster culture conditions, embryo culture or an academic research relating a survival environment.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a schematic diagram of a cell culture realtime observation system according to an embodiment of the present invention;

FIG. 2 is a schematic diagram of a cell culture realtime observation system according to a first embodiment of the present invention;

FIG. 3 is a schematic diagram of a cell culture realtime observation system according to a second embodiment of the present invention;

FIG. 4 is a schematic diagram of a cell culture realtime observation system according to a third embodiment of the present invention;

FIG. 5 is a schematic diagram of a cell culture realtime observation system according to a fourth embodiment of the present invention;

FIG. 6 is a schematic diagram of a cell culture realtime observation system according to a sixth embodiment of the present invention; and

FIG. 7 is a schematic diagram of a cell culture realtime observation system according to a seventh embodiment of the present invention.

### DETAILED DESCRIPTION OF THE PREFERRED EMBODIMENTS

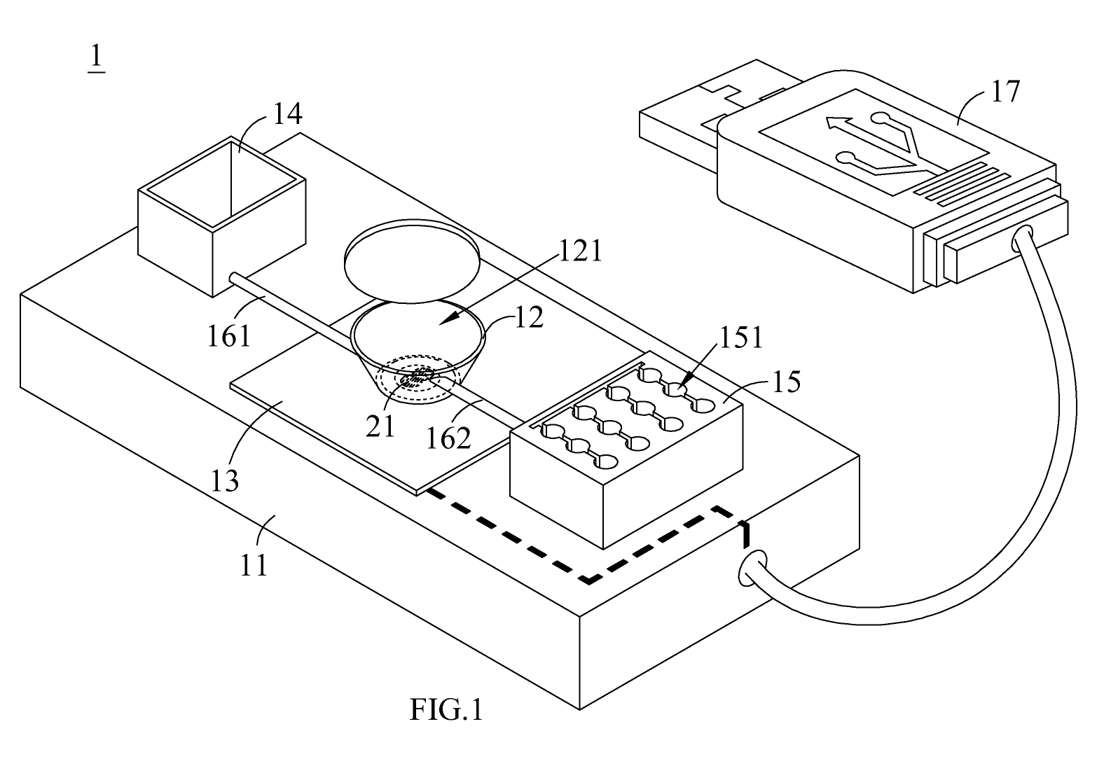

Pleased refer to FIG. 1, which is a schematic diagram of a cell culture realtime observation system according to an embodiment of the present invention. As shown, the cell culture realtime observation system comprises a substrate 11, a culture well 12, an image capture device 13, at least one container 14, an evaporation device 15, a first micro-channel 161, a second micro-channel 162 and a transmitting unit 17. The culture well 12 is disposed on the substrate 11 and has an opening capable of closing 121, and the opening capable of closing 121 may be disposed not only at the top of the culture well 12 but also around the culture well 12. The opening 121 may be a cover type opening, a dragging type opening or a rolling type opening. The image capture device 13 is disposed between the substrate 11 and the culture well 12. The at least one container 14 is disposed on the substrate 11 and at a side of the culture well 12. The evaporation device 15 is disposed on the substrate 11 and at the other side of the culture well 12, and a surface of the evaporation device 15 has a plurality of evaporation holes 151. The first micro-channel 161 connects the at least one container 14 to the culture well 12. The second micro-channel 162 connects the culture well 12 to the evaporation device 15. The transmission unit 17 is electrically connected to the image capture device 13. Additionally, the first micro-channel 161 is disposed at the bottom of the culture well 12 through a side of the container 14, and the second micro-channel 162 is disposed at the upside of the culture well 12 and then connected to the evaporation device 15.

A cell 21, such as an embryo, is placed in the culture well 12 by a user, and a culture medium is contained within at least one container 14. The culture medium is flowed through the at least one container 14, the culture well 12 and the evaporation device 15 by the first micro-channel 161 and the second micro-channel 162 to immerse the cell 21 in the culture medium. When the culture medium is flowed into the evaporation device 15, the culture medium is evaporated through the plurality of evaporation holes 151 so as to generate an evaporative driving force. The culture medium may be flow slowly on the first micro-channel 161 and the second micro-channel by the evaporative driving force. When the image of the cell 21 is captured by the image capture device 13, the captured image can be transmitted to an electronic device 31 through the transmitting unit 17. Thus, a user can observe the cell 21 in real time during cultivation.

The image capture device 13 may be a charge-coupled device (CCD) image capture device or a complementary metal oxide semiconductor (CMOS) image capture device, and the transmission unit 17 may be a USB port. The culture well 12 and the container 14 must be made of biocompatibility materials.

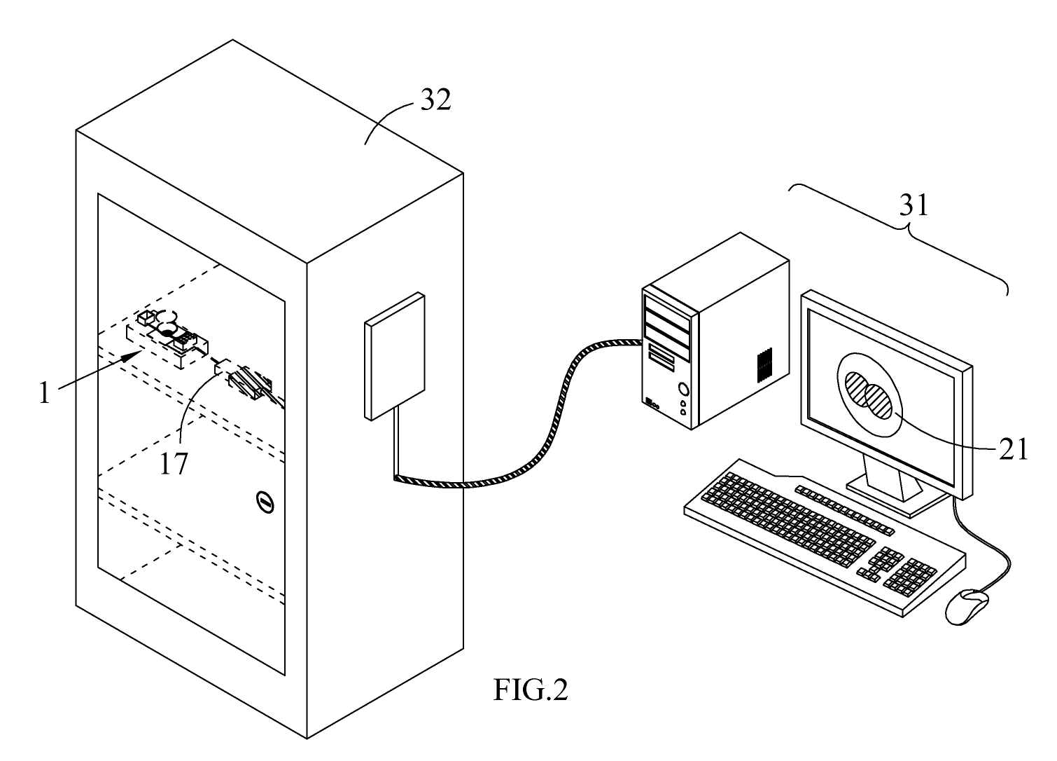

Please refer to FIG. 2, which is a schematic diagram of a cell culture realtime observation system according to a first embodiment of the present invention. As shown, the cell culture realtime observation system 1 is disposed in an incubator 32, and the cell image captured by the image capture device 13 can be transmitted to the electronic device 31, such as a computer, through the transmitting unit 17. The number of taking pictures can be controlled by the computer, such that the cell morphology can be detected in real time, and the development process of cells or embryos can be observe for a long time.

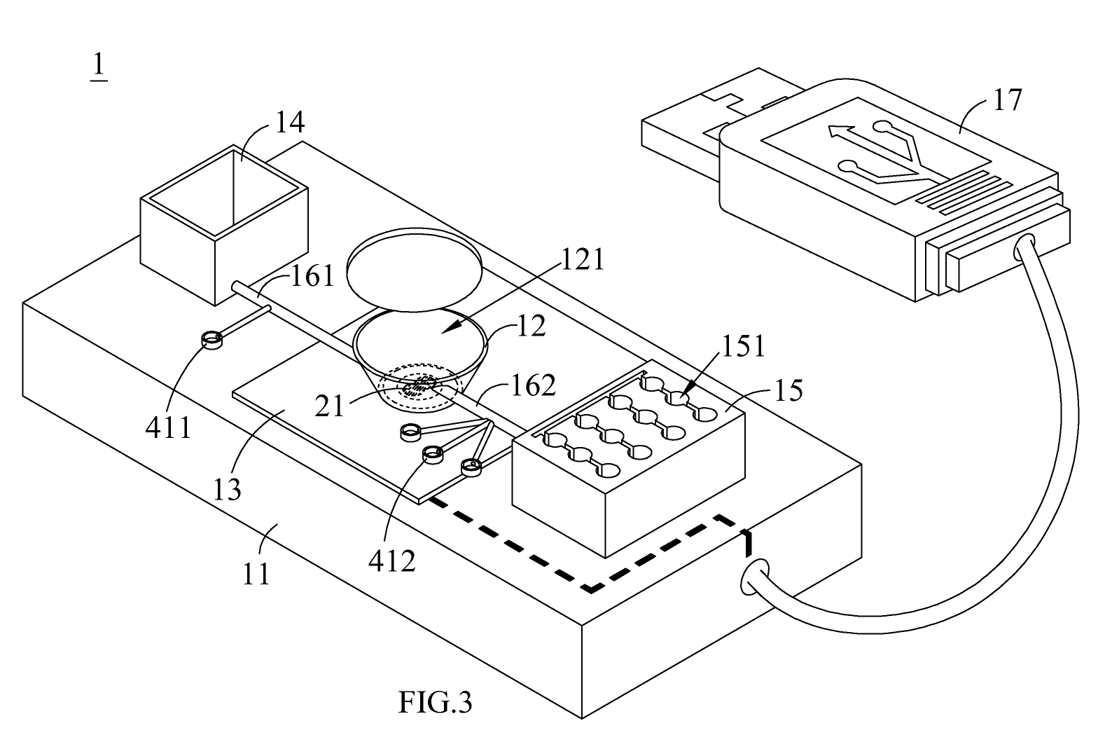

Furthermore, the cell culture realtime observation system 1 may further comprise a first diverging channel 411 and a second diverging channel 412. The first diverging channel 411 is disposed on the first micro-channel 161 and the second diverging channel 412 is disposed on the second micro-channel 162, as shown in FIG. 3. At least one drug or agent can be contained on the first diverging channel 411 to mix with the culture medium, and both of the culture medium and the at least one drug or agent are flowed into the bottom of the culture well 12 to immerse the cell 21. After the cell 21 is treated with the drug or agent, the cell 21 will release substances, such as antioxidants or toxicants, to the culture medium. The culture medium, which includes the substances released from the cell, is collected via the second diverging channel 412 to detect the substances. Whether the quality of the culture medium is altered to generate toxicants or not is also detected.

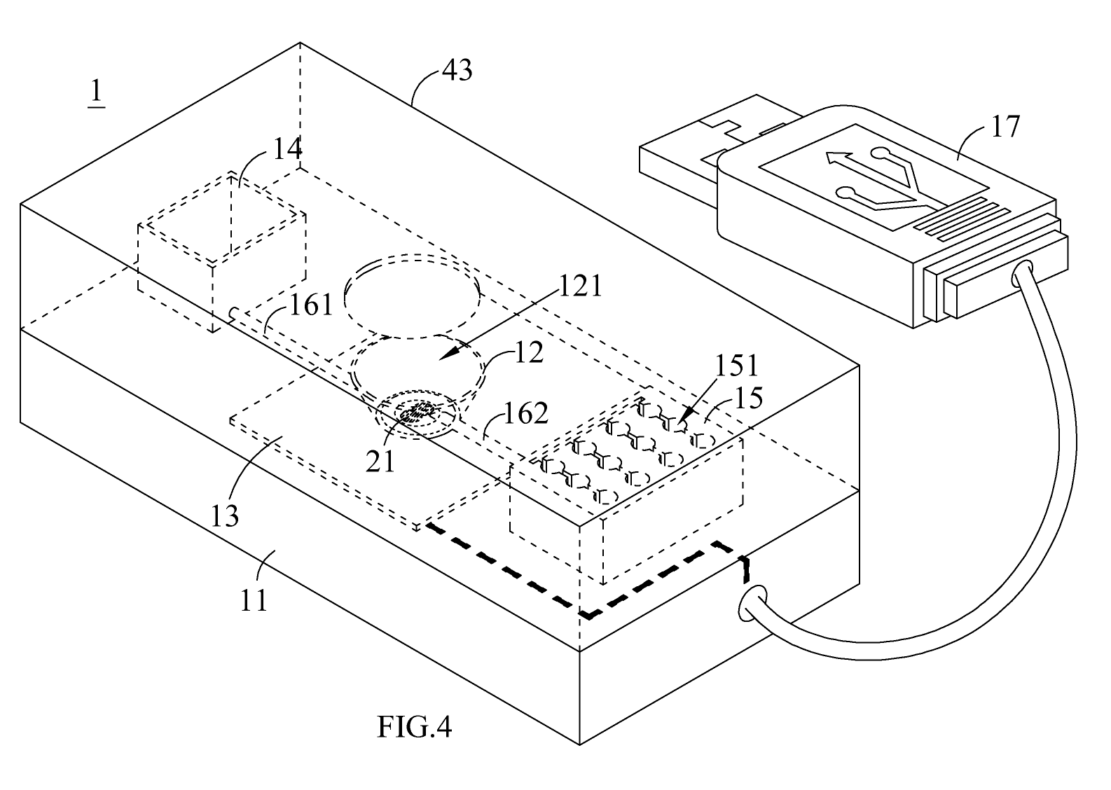

The cell culture realtime observation system 1 may comprise a package mechanism 43. The culture well 12, the image capture device 13, the at least one container 14, the evaporation device 15, the first micro-channel 161 and the second micro-channel 162 are packaged within the package mechanism 43, as shown in FIG. 4. The transmission unit 17 is disposed at an end of the package mechanism 43 for transmitting the image to the electronic device 31. The package mechanism 43 may be a satirizing-type package mechanism, that is, the package mechanism can be sterilized by immersing in ethanol, an ethylene oxide (E.O.) sterilization, a radiation sterilization or an ozone sterilization, and therefore all cell contaminations resulted from environment and user factors during cell culture can be prevented. The package mechanism 43 can be made of transparent material for observing whether the culture medium is enough or not in real time. In another embodiment, the package mechanism 43 and the substrate 11 can be unitarily formed, and the opening capable of closing 121 is disposed at a side of the package mechanism 43 corresponding to the culture well 12. Therefore, the user can put the cell 21 onto the culture well 12 without opening the package mechanism 43.

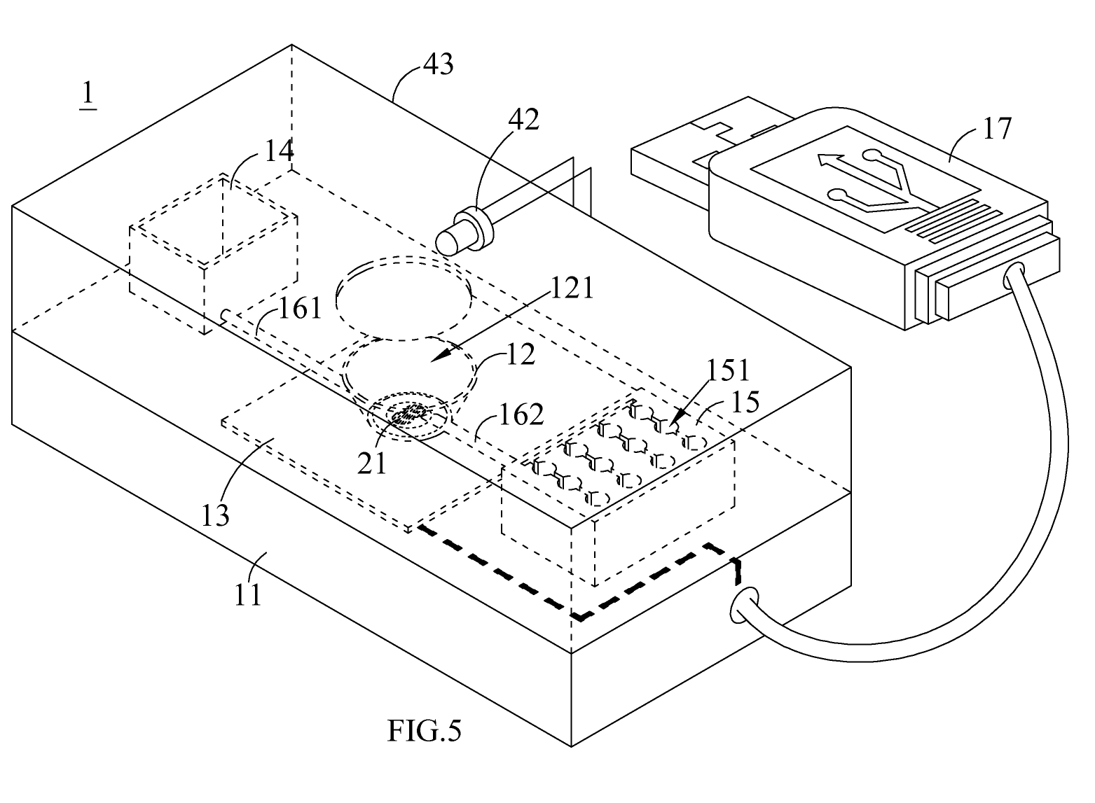

Additionally, a light source device 42 may be disposed on the package mechanism 43 in the cell culture realtime observation system 1 of the present invention, as shown in FIG. 5. The light source device 42 may be a LED source without phototoxicity, and the culture well 12 can be lighted up by the light radiated therefrom. The light source device 42 may radiate collimating rays or non-collimating rays, which can light up the cell 21 while taking pictures under threshold temperature and do not affect the development and growth of the cell 21 or an organism to be detected. The optical heating resulted from the focus-lighting of microscopes at parts of the cell 21 can be prevented. Further, the development rate of the cells can be increase or decrease by different light wavelength or irradiation conditions from the light source device.

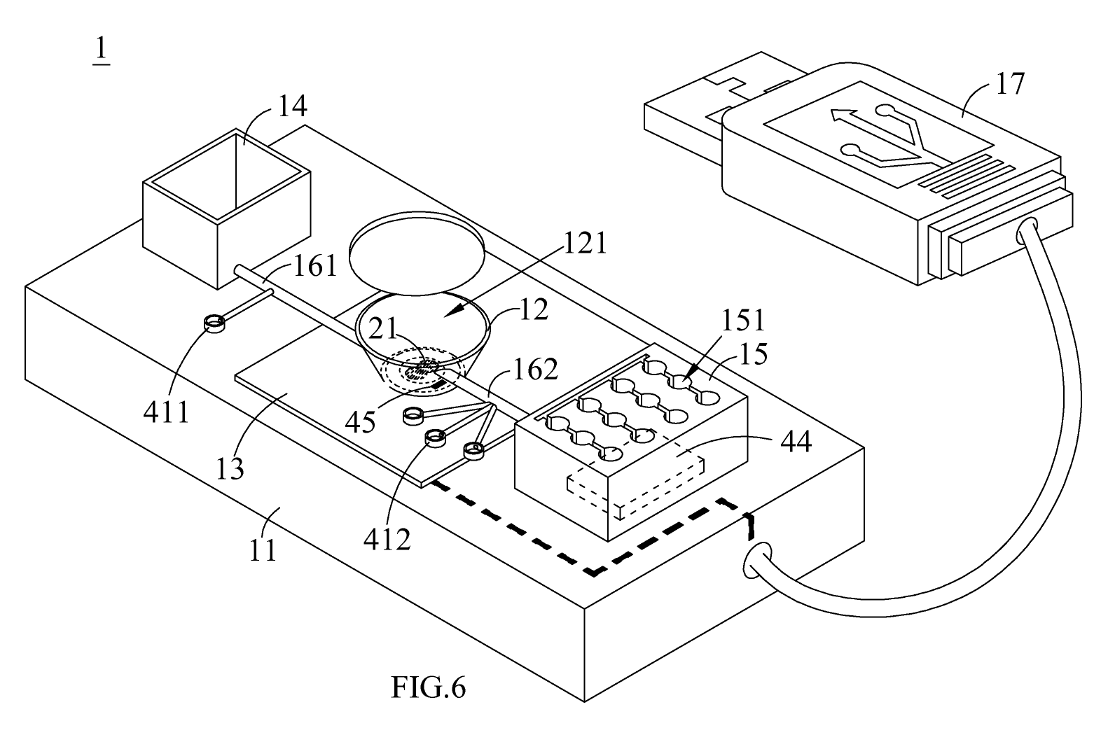

Because the evaporation device 15 of the cell culture realtime observation system 1 in the present invention has the plurality of evaporation holes 151, a user can control the evaporative driving force of the culture medium by controlling the sizes of the plurality of evaporation holes to stabilize the velocity and hydraulic pressure of the culture medium and to prevent from fluid shear stress thereof in any micro channels. Thus, it is unnecessary to dispose expensive mechanical and microelectromechanical (MEM) pumps. Nevertheless, if the evaporation velocity of the culture medium should be increased due to experiment needs, a temperature controller 44 may be disposed under the evaporation device 15 and a temperature sensor 45 may be disposed in the culture well 12 to simultaneously detect the temperature of the cell 21 and the culture medium, as shown in FIG. 6. In addition, the means of controlling temperatures indicate that the temperature of the evaporation device 15 can be controlled by proximal control or remote control. The proximal control and the remote control mean that the temperature of the evaporation device 15 can be controlled by the temperature controller 44 disposed in the cell culture realtime observation system of the present invention and the electronic device 31, respectively. When a user or a feedback control system intends to increase the evaporation velocity, the temperature of the evaporation device 15 can be increased by the temperature controller 44 or the electronic device 31. On the other hand, when the temperature of the evaporation device 15 is very high, the user can control the temperature controller 44 to decrease the temperature for achieving the cooling purposes. The temperature controller 44 in the evaporation device 15 and the temperature sensor 45 in the culture well 12 are also used for adjusting temperatures. Accordingly, the temperature sensor 45 can detect and adjust the temperature of the culture well 12, and the temperature controller 44 in the evaporation device 15 can be used for detect and adjust the evaporation rate. Ions crystallized at outside of the first micro-channel or the second micro-channel can also be determined to control the ion concentration of the culture medium.

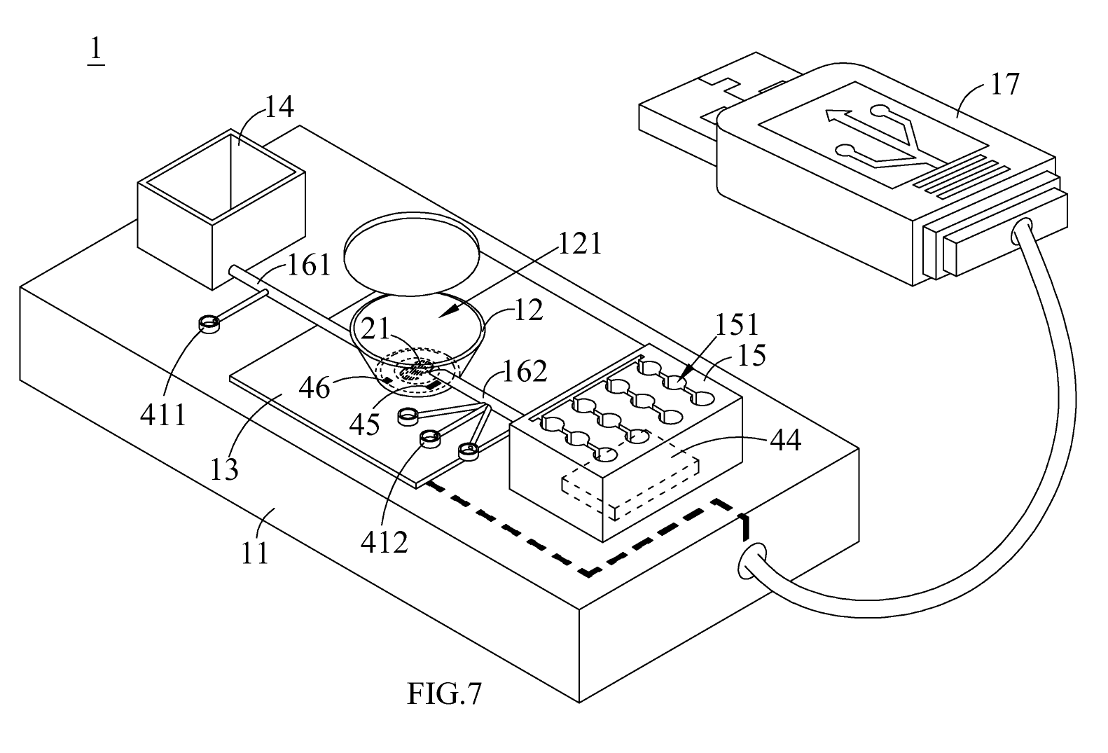

In the cell culture realtime observation system 1 of the present invention, a potential sensor 46 may be disposed in the culture well 12 for detecting the variation of the culture medium, as shown in FIG. 7. For example, when a cell 21 is under a high oxidative pressure environment, the oxidative stress of the cell 21 may be increased to generate reactive oxygen species (ROS), such as superoxide anions to release into the culture medium. Therefore, the potential sensor 46 can detect whether the cells is under the high oxidative pressure environment or not. The change of the concentrations of ions, such as calcium or potassium, in the culture medium can also be detected by the potential sensor 46 to maintain the equilibrium of the ions in the culture medium.

The description as set forth, because the image capture device combined with the culture well in the cell culture realtime observation system of the present invention, a user can record the status of a cell during cultivation in real time. The user does not need to move cells frequently, such that the cell can be cultured under stable conditions. Additionally, the transmission unit may be a USB port to get operated easily at first time for users.

The present invention has been described with some preferred embodiments thereof, and it is understood that many changes and modifications in the described embodiments can be carried out without departing from the scope and the spirit of the invention that is intended to be limited only by the appended claims.

What is claimed is:

1. A cell culture realtime observation system, comprising:
a substrate;
a culture well disposed on the substrate for placing a cell in the culture well;
an image capture device disposed between the substrate and the culture well to capture an image of the cell;
at least one container disposed on the substrate and at a side of the culture well for containing a culture medium;
an evaporation device disposed on the substrate and at another side of the culture well, and a surface of the evaporation device having a plurality of evaporation holes;
a first micro-channel connecting the at least one container to the culture well;
a second micro-channel connecting the culture well to the evaporation device; wherein the culture medium is flowed through the at least one container, the culture well and the evaporation device via the first micro-channel and the second micro-channel to immerse the cell in the culture medium; when the culture medium is flowed into the evaporation device, the culture medium generates an evaporative driving force and is flowed slowly on the first micro-channel and the second micro-channel; and
a transmission unit electrically connected to the image capture device and transmitting the image captured by the image capture device to an electronic device for observing the cell in realtime by a user.

2. The cell culture realtime observation system as claimed in claim 1, further comprising:
a first diverging channel disposed on the first micro-channel and containing at least one drug or agent; and
a second diverging channel disposed on the second micro-channel for collecting the culture medium treated with the at least one drug or agent.

3. The cell culture realtime observation system as claimed in claim 1, further comprising a package mechanism; the culture well, the image capture device, the at least one container, the evaporation device, the first micro-channel and the second micro-channel packaged within the package mechanism; and the transmission unit disposed at an end of the package mechanism for transmitting the image to the electronic device.

4. The cell culture realtime observation system as claimed in claim 3, further comprising a light source device disposed on the package mechanism corresponding to the culture well, and the light source device radiating light to the cell.

5. The cell culture realtime observation system as claimed in claim 1, further comprising a potential sensor disposed in the culture well for detecting the variation of the culture medium.

6. The cell culture realtime observation system as claimed in claim 1, further comprising a temperature controller disposed under the evaporation device for controlling an evaporation velocity of the culture medium.

7. The cell culture realtime observation system as claimed in claim 6, further comprising a temperature sensor disposed in the culture well for detecting a temperature of the culture medium containing with the cell.

8. The cell culture realtime observation system as claimed in claim 1, wherein the image capture device comprises a charge-coupled device (CCD) image capture device or a complementary metal oxide semiconductor (CMOS) image capture device.

9. The cell culture realtime observation system as claimed in claim 1, wherein the transmission unit comprises a USB port.
